(19) United States
(12) Patent Application Publication
(10) Pub. No.: US 2023/0381109 A1
(43) Pub. Date: Nov. 30, 2023

(54) ORALLY DISINTEGRATING TABLET COMPRISING BENZIMIDAZOLE DERIVATIVE COMPOUND AND PREPARATION METHOD THEREOF

(71) Applicant: HK INNO.N CORPORATION, Chungcheongbuk-do (KR)

(72) Inventors: Min Jung Kim, Gyeonggi-do (KR); Sun Young Park, Gyeonggi-do (KR); Da Som Lim, Gyeonggi-do (KR); Eun Kyung Jeon, Gyeonggi-do (KR); Young Dae Cho, Gyeonggi-do (KR); Tae Keun Cho, Incheon (KR)

(21) Appl. No.: 18/032,749

(22) PCT Filed: Oct. 21, 2021
(86) PCT No.: PCT/KR2021/014853

§ 371 Date: Apr. 19, 2023

(30) Foreign Application Priority Data
Oct. 23, 2020 (KR) 10-2020-0138712

(51) Int. Cl. A61K 9/20; A61K 31/4184; A61K 9/00; A61P 1/04

(52) U.S. Cl. CPC A61K 9/20; A61K 31/4184; A61K 9/2018; A61K 9/205; A61K 9/2054; A61K 9/2059; A61K 9/2009; A61K 9/2013; A61K 9/2095; A61K 9/0056; A61P 1/04

(57) ABSTRACT

The present invention relates to an orally disintegrating tablet including a benzimidazole derivative compound and a preparation method thereof.

### TECHNICAL FIELD

The present invention relates to an orally disintegrating tablet including a benzimidazole derivative compound and a preparation method thereof and, more specifically, to an orally disintegrating tablet containing wet granules including tegoprazan, a pharmaceutically acceptable salt thereof, a hydrate or solvate thereof, or a mixture thereof; and a sweetening agent, and a preparation method thereof.

### BACKGROUND ART

Tegoprazan is a compound named
(S)-4-(5,7-difluorochroman-4-yloxy)-N,N,2-trimethyl-1H-benzo[d]imidazole-6-carbox amide (or
7-{[(4S)-5,7-difluoro-3,4-dihydro-2H-chromen-4-yl]oxy }-N,N,2-trimethyl-1H-benzim idazole-5-carboxamide) and is a potassium-competitive acid blocker (P-CAB) having a mechanism similar to that of an acid pump antagonist (APA). This competes with potassium ions in binding to H⁺/K⁺-ATPase (proton pump), an enzyme that secretes H⁺ ions, a component of gastric acid in parietal cells of the stomach, into the gastric lumen, thereby inhibiting gastric acid secretion.

Tegoprazan is used as a therapeutic agent for gastroesophageal reflux disease, and at least 70% of patients with gastroesophageal reflux disease are the elderly in their 60s or older who have difficulty in deglutition. Many of those patients have less ability to swallow, and thus have many difficulties in taking oral dosage forms such as tablets, capsules and the like.

An orally disintegrating tablet is convenient for those patients to take, but tegoprazan is a drug that exhibits a bitter taste even at low concentrations, and thus there is also a limit to the development of tegoprazan into the orally disintegrating tablet.

Accordingly, there is a need to develop an orally disintegrating tablet that disintegrates in the oral cavity within a short time while exhibiting excellent sensory properties with a masked bitter taste of tegoprazan.

### RELATED ART REFERENCE

### Patent Document

International Patent Publication WO 2007/072146

### DISCLOSURE OF INVENTION

### Technical Problem

The present invention may provide an orally disintegrating tablet containing wet granules including tegoprazan, a pharmaceutically acceptable salt thereof, a hydrate or solvate thereof, or a mixture thereof; and a sweetening agent. The present invention may provide a method for preparing an orally disintegrating tablet, the method including: (1) preparing wet granules including tegoprazan, a pharmaceutically acceptable salt thereof, a hydrate or solvate thereof, or a mixture thereof and a sweetening agent; (2) preparing a mixture by adding one or more pharmaceutically acceptable additives to the wet granules; and (3) compressing the mixture into tablets.

The present invention may provide an orally disintegrating tablet prepared by the above preparation method.

### Solution to Problem

Terms not specifically defined in the present specification should be understood to have meanings commonly used in the art to which the present invention pertains. In addition, singular forms include plural forms and also vice versa unless otherwise defined by context.

In the present specification, items are arbitrarily divided for convenience of description of the specification, and the content of any one item should not be construed as dependent on the item.

Orally Disintegrating Tablet

The present invention may provide an orally disintegrating tablet containing wet granules including a compound represented by formula 1 below, a pharmaceutically acceptable salt thereof, a hydrate or solvate thereof, or a mixture thereof; and a sweetening agent:

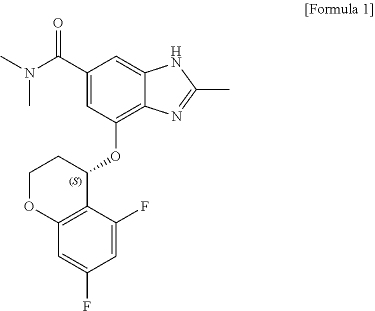

The compound represented by formula 1 above is named
(S)-4-(5,7-difluorochroman-4-yloxy)-N,N,2-trimethyl-1H-benzo[d]imidazole-6-carbox amide, and is also called tegoprazan.

In the present invention, the “pharmaceutically acceptable salt” may refer to the salts

formed with any inorganic acid, organic acid or base, which neither causes a serious stimulus to the subject dosed therewith, nor does damage to biological activity and physical property of the tegoprazan. Salts used herein may include the salts conventionally used in the art, such as acid-addition salts formed with pharmaceutically acceptable free acid. The pharmaceutically acceptable salt may be specifically selected from the group consisting of pidolate salt, acetate salt, adipate salt, aspartate salt, benzoate salt, besylate salt, bicarbonate salt/carbonate salt, bisulfate salt/sulfate salt, borate salt, camsylate salt, citrate salt, cyclamate salt, edisylate salt, esylate salt, formate salt, fumarate salt, gluceptate salt, gluconate salt, glucuronate salt, hexafluorophosphate salt, hibenzate salt, hydrochloride salt/chloride salt, hydrobromide salt/bromide salt, hydroiodide salt/iodide salt, isethionate salt, lactate salt, malate salt, maleate salt, malonate salt, mesylate salt, methylsulphate salt, naphthylate salt, 2-napsylate salt, nicotinate salt, nitrate salt, orotate salt, palmitate salt, pamoate salt, phosphate salt/hydrogen phosphate salt/dihydrogen phosphate salt, pyroglutamate salt, saccharate salt, stearate salt, succinate salt, tannate salt, tartrate salt, tosylate salt, trifluoroacetate salt and xinofoate salt, but are not limited thereto. Any salts may be used without limitation, as long as they may conventionally show the pharmacological activity of said tegoprazan.

In the present invention, the “hydrate” may refer to one in which tegoprazan or a pharmaceutically acceptable salt thereof and water are bound by a non-covalent intermolecular force, and may include a stoichiometric or non-stoichiometric amount of water.

In the present invention, the “solvate” may refer to one in which tegoprazan or a pharmaceutically acceptable salt thereof and a solvent other than water are bound by a non-covalent intermolecular force, and may include a stoichiometric or non-stoichiometric amount of the solvent.

In the present specification, tegoprazan may refer to a compound represented by formula 1 above, as well as a pharmaceutically acceptable salt thereof, a hydrate or solvate thereof, or a mixture thereof all.

In the present invention, tegoprazan may be present in a crystalline or amorphous form.

The orally disintegrating tablet of the present invention may contain wet granules and the wet granules may include a compound represented by above formula 1, a pharmaceutically acceptable salt thereof, a hydrate or solvate thereof, or a mixture thereof as an active ingredient.

The orally disintegrating tablet of the present invention may be used in the treatment of diseases mediated by an acid pump antagonistic activity due to its properties in that the tablet includes the compound represented by formula 1 above, a pharmaceutically acceptable salt thereof, a hydrate or solvate thereof, or a mixture thereof.

The diseases mediated by an acid pump antagonistic activity may be at least one selected from the group consisting of gastrointestinal disease, gastroesophageal disease, gastroesophageal reflux disease (GERD), peptic ulcer, gastric ulcer, duodenal ulcer, NSAID-induced ulcer, gastritis, Helicobacter pylori infection, dyspepsia, functional dyspepsia, Zollinger-Ellison syndrome, nonerosive reflux disease (NERD), visceral referred pain, purosis, nausea, esophagitis, dysphagia, salivation, airway lesion and asthma, and specifically may be gastroesophageal reflux disease (GERD), but are not limited thereto.

The “gastroesophageal reflux disease (GERD)” may refer to a case in which gastric contents reflux into the esophagus, causing symptoms that interfere with daily life or causing complications, and may be divided into erosive esophagitis (EE) and non-erosive reflux disease (NERD).

The orally disintegrating tablet of the present invention may include a therapeutically effective amount of tegoprazan.

In the present invention, the “therapeutically effective amount” may refer to an amount effective in preventing or treating the diseases mediated by an acid pump antagonistic activity.

The orally disintegrating tablet of the present invention may include wet granules, and the wet granules may include a sweetening agent.

In the present invention, the wet granules may include the compound represented by formula 1, a pharmaceutically acceptable salt thereof, a hydrate or solvate thereof, or a mixture thereof, and a sweetening agent at a weight ratio of 1:0.001 to 1:0.4.

The compound represented by formula 1, a pharmaceutically acceptable salt thereof, a hydrate or solvate thereof, or a mixture thereof and a sweetening agent may be included specifically at a weight ratio of 1:0.001 to 1:0.4, 1:0.005 to 1:0.35, and 1:0.01 to 1:0.3, more specifically 1:0.05 to 1:0.2, but is not limited thereto.

In this regard, in one specific embodiment of the present invention, it was confirmed that a less total amount of related substances is generated, thereby exhibiting excellent stability, if the orally disintegrating tablet of the present invention includes the compound represented by formula 1, a pharmaceutically acceptable salt thereof, a hydrate or solvate thereof, or a mixture thereof and a sweetening agent at a weight ratio of 1:0.05 to 1:0.2 (Table 8).

In the present invention, the sweetening agent may be any one selected from the group consisting of sucralose, aspartame, saccharin, acesulfame potassium, stevioside, enzymatically modified stevia, sucrose, isomalt, maltitol, mannitol, sorbitol, steviol glycoside, erythritol, xylose, xylitol, lactitol, neotame, ribose, tomatine, polyglycitol, advantam, tagatose, trehalose, glucose, maltose, dextrose, white sugar, fructose, honey, glycyrrhizin, monellin, rubusoside, curculin, corn syrup, lactose, oligosaccharides, and mixtures thereof, and specifically may be sucralose, aspartame or maltitol, but is not limited thereto.

In this regard, in one specific embodiment of the present invention, it was confirmed that the orally disintegrating tablet of the present invention generates a less total amount of related substances, thereby exhibiting excellent stability regardless of the type of the sweetening agent (Table 8).

In the present invention, the sweetening agent may be included in an amount of 0.01 to 10 wt %, specifically 0.01 to 10 wt %, 0.01 to 8 wt %, 0.07 to 6 wt %, 0.1 to 4 wt %, more specifically 0.1 to 3 wt %, and much more specifically 0.7 to 2 wt % based on the total weight of the tablet, but is not limited thereto.

The orally disintegrating tablet of the present invention may further include pharmaceutically acceptable additives.

The “pharmaceutically acceptable additives” may refer to any additives that neither cause a serious stimulus to the subject dosed therewith, nor do damage to biological activity and physical property of the tegoprazan, and may specifically include excipients, disintegrants, diluents, flavoring agents, sweetening agents, lubricants, etc., but are not limited thereto.

The orally disintegrating tablet of the present invention may further include a disintegrant as pharmaceutically acceptable additives.

In the present invention, the disintegrant may be any one selected from the group consisting of crospovidone, croscarmellose sodium, sodium starch glycolate, alginic acid, sodium alginate, low-substituted hydroxypropyl cellulose, carboxymethyl cellulose, corn starch, pregelatinized starch, microcrystalline cellulose, hydroxypropyl cellulose, sodium bicarbonate, and mixtures thereof, and specifically may be crospovidone, but is not limited thereto.

When crospovidone is used as the disintegrant, a size of the particle may be specifically 10 to 130 μcm and more specifically 10 to 40 μcm, but is not limited thereto.

In the present invention, the disintegrant may be included in an amount of 1 to 50 wt %, specifically 1 to 50 wt %, 1 to 40 wt %, 1 to 30 wt %, 1 to 20 wt %, 1 to 10 wt %, and more specifically 2 to 7 wt % based on the total weight of the tablet, but is not limited thereto.

The orally disintegrating tablet of the present invention may further include diluents, flavoring agents, sweetening agents, lubricants, or mixtures thereof as pharmaceutically acceptable additives.

In the present invention, the diluent may be any one selected from the group consisting of mannitol, lactose, starch, microcrystalline cellulose, ludipress (BASF), pearlitol flash (Roqutte), calcium dihydrogen phosphate, dextrose, maltose, erythritol, sucrose, maltitol, trihalose, glucose, xylitol, F-melt (Fujichemical), sorbitol, pre-gelatinized starch, anhydrous calcium hydrogen phosphate, dicalcium phosphate, and mixtures thereof, and specifically may be mannitol, xylitol, pearlitol flash or mixtures thereof, but is not limited thereto.

In the present invention, the diluent may be included in an amount of 1 to 99 wt %, specifically 1 to 99 wt %, 5 to 97 wt %, 10 to 95 wt %, 15 to 92 wt %, and more specifically 20 to 90 wt % based on the total weight of the tablet, but is not limited thereto.

In the present invention, the flavoring agent may be any one selected from the group consisting of peppermint flavor, yogurt flavor, fruit flavor, and mixtures thereof, and specifically may be peppermint flavor, but is not limited thereto. The fruit flavor may be specifically apple flavor, grape flavor, strawberry flavor, or lemon flavor, but is not limited thereto.

In the present invention, the flavoring agent may be included in an amount of 0.01 to 10 wt %, specifically 0.05 to 8 wt %, 0.1 to 6 wt %, 0.2 to 4 wt %, and more specifically 0.3 to 3 wt % based on the total weight of the tablet, but is not limited thereto.

The orally disintegrating tablet of the present invention may further include a sweetening agent in addition to the sweetening agent included in the wet granules, and the sweetening agent to be further included may be any one selected from the group consisting of sucralose, aspartame, saccharin, acesulfame potassium, stevioside, enzymatically modified stevia, sucrose, isomalt, maltitol, mannitol, sorbitol, steviol glycoside, erythritol, xylose, xylitol, lactitol, neotame, ribose, tomatine, polyglycitol, advantam, tagatose, trehalose, glucose, maltose, dextrose, white sugar, fructose, honey, glycyrrhizin, monellin, rubusoside, curculin, corn syrup, lactose, oligosaccharides, and mixtures thereof, and specifically may be maltitol, sucralose, enzymatically modified stevia or mixtures thereof, but is not limited thereto.

The sweetening agent included in the wet granules and the sweetening agent further included therein may be of the same type or different types.

In the present invention, the sweetening agent included in the wet granules may be referred to as a first sweetening agent, and the sweetening agent further included therein may be referred to as a second sweetening agent. In this case, “first,” “second,” etc. are only used to distinguish a plurality of components, and do not indicate priority thereof.

The orally disintegrating tablet of the present invention may include the sweetening agent in an amount of 0.01 to 10 wt %, specifically 0.01 to 5 wt %, and more specifically 0.1 to 3 wt % based on the total weight of the tablet without distinction between the sweetening agent included in the wet granules and the sweetening agent further included therein, but is not limited thereto.

In the present invention, the lubricants may be any one selected from the group consisting of stearic acid, stearic acid metal salts, talc, colloidal silicon dioxide, sucrose fatty acid ester, hydrogenated vegetable oil, wax, glycerin fatty acid ester, glycerol dibehenate, and mixtures thereof, and specifically may be stearic acid metal salts, colloidal silicon oxide, or mixtures thereof, but are not limited thereto. The stearic acid metal salts may be more specifically calcium stearate or magnesium stearate, but are not limited thereto.

In the present invention, the lubricants may be included in an amount of 0.1 to 10 wt %, specifically 0.1 to 10 wt %, 0.2 to 8 wt %, 0.3 to 7 wt %, 0.4 to 6 wt %, and more specifically 0.5 to 5 wt % based on the total weight of the tablet, but are not limited thereto.

In the present invention, the wet granules may be prepared by high-speed shear granulation or fluidized bed granulation, but are not limited thereto.

The wet granules may include ones prepared by gathering powder particles with a granulation fluid (binder solution), and the granulation fluid may be used alone or in combination with a binder or an additive capable of giving binding force in order to impart adhesion between particles in a dry state.

In the present invention, the wet granules may be prepared with a binder solution including any one selected from the group consisting of alcohol, water, and a mixture thereof and a sweetening agent.

In the wet granules prepared with the binder solution including the sweetening agent, particles of tegoprazan and excipients may be physically closely bound in the granules, i.e., may be subjected to attachment and coating.

In the present invention, the binder solution may further include a binder or an additive capable of giving binding force.

The binder or the additive capable of giving binding force may be any one selected from the group consisting of hydroxypropylmethylcellulose (HPMC), hydroxypropylcellulose (HPC), gelatin, pregelatinized starch, polyvinylpyrrolidone, polyvinyl alcohol, pullulan, polyethylene glycol, natural gum, synthetic gum, copovidone, ethyl cellulose, methacrylate copolymer, and mixtures thereof, and specifically may be hydroxypropylmethylcellulose, but is not limited thereto.

The binder or the additive capable of giving binding force may be added as a solution or as a dry material mixed with primary powder particles.

In this regard, in one specific embodiment of the present invention, the wet granules were prepared with a binder solution including an aqueous solution of 58% (w/w) ethanol and sucralose; a binder solution including an aqueous solution of 50% (w/w) ethanol and sucralose; a binder solution including an aqueous solution of 50% (w/w) ethanol, hydroxypropylmethylcellulose and sucralose; a binder solution including an aqueous solution of 50% (w/w) ethanol and maltitol; or a binder solution including an aqueous solution of 50% (w/w) ethanol and aspartame.

The oral disintegrating tablet of the present invention may disintegrate rapidly within 30 seconds.

In this regard, in one specific embodiment of the present invention, it was confirmed that the orally disintegrating tablet of the present invention disintegrates within 30 seconds in an in vitro disintegration experiment, an in vivo disintegration experiment and a disintegration experiment in a disintegration tester (Table 2).

The orally disintegrating tablet of the present invention may exhibit excellent sensory properties with a masked bitter taste.

In this regard, in one specific embodiment of the present invention, it was confirmed that the orally disintegrating tablet of the present invention has a very little bitter taste and a feeling of soft disintegration in the oral cavity, thereby being effective in ameliorating the bitter taste and exhibiting excellent sensory properties (Table 3).

The orally disintegrating tablet of the present invention may disintegrate in the oral cavity within a short time and exhibit excellent sensory properties such as a masked bitter taste, etc., thereby greatly improving a patient's compliance with medication. Thus, the orally disintegrating tablet of the present invention may be effective in treating patients with gastroesophageal reflux disease, particularly patients with gastroesophageal reflux disease, who have difficulty in deglutition.

The orally disintegrating tablet of the present invention may show an excellent dissolution rate.

In this regard, in one specific embodiment of the present invention, it was confirmed that the orally disintegrating tablet of the present invention exhibits an excellent dissolution rate of tegoprazan at a level similar to that of the reference drug K-CAP tablet without delay in release (Table 4, FIGS. 1 and 2).

The orally disintegrating tablet of the present invention may show excellent stability.

In this regard, in one specific embodiment of the present invention, it was confirmed that the orally disintegrating tablet of the present invention generates a less total amount of related substances under the stress and accelerated conditions, thereby exhibiting excellent stability (Table 6).

In this regard, in one specific embodiment of the present invention, it was confirmed that the orally disintegrating tablet of the present invention exhibits excellent stability in appearance without any change in appearance caused by moisture and heat on the surface of the tablet under the stress and accelerated conditions (FIG. 3).

Method for Preparing Orally Disintegrating Tablet

The present invention may provide a method for preparing an orally disintegrating tablet, which includes: (1) preparing wet granules including a compound represented by formula 1 below, a pharmaceutically acceptable salt thereof, a hydrate or solvate thereof, or a mixture thereof, and a sweetening agent;
(2) preparing a mixture by adding one or more pharmaceutically acceptable additives to the wet granules; and
(3) compressing the mixture into tablets:

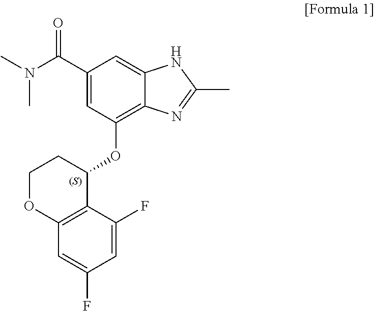

In the method for preparing an orally disintegrating tablet of the present invention, the contents described in the foregoing item of the orally disintegrating tablet may be equally applied as long as there is no contradiction. Thus, the wet granules, the components included therein, the content of respective components, the weight ratio of respective components, uses, effects, etc. of the foregoing orally disintegrating tablet may be directly applied to the method for preparing the orally disintegrating tablet as long as there is no contradiction.

In the present invention, the sweetening agent may be any one selected from the group consisting of sucralose, aspartame, saccharin, acesulfame potassium, stevioside, enzymatically modified stevia, sucrose, isomalt, maltitol, mannitol, sorbitol, steviol glycoside, erythritol, xylose, xylitol, lactitol, neotame, ribose, tomatine, polyglycitol, advantam, tagatose, trehalose, glucose, maltose, dextrose, white sugar, fructose, honey, glycyrrhizin, monellin, rubusoside, curculin, corn syrup, lactose, oligosaccharides, and mixtures thereof, and specifically may be sucralose, aspartame or maltitol, but is not limited thereto.

In the present invention, the preparing wet granules may be perfomed by a wet granulation with a binder solution including any one selected from the group consisting of alcohol, water, and a mixture thereof and a sweetening agent. The binder solution may specifically include a mixture of alcohol and water, more specifically an aqueous solution of ethanol, and much more specifically an aqueous solution of 58% (w/w) ethanol or an aqueous solution of 50% (w/w) ethanol, but is not limited thereto.

In the present invention, the binder solution may further include a binder or an additive capable of giving binding force.

In the present invention, the binder or the additive capable of giving binding force may be any one selected from the group consisting of hydroxypropylmethylcellulose (HPMC), hydroxypropylcellulose (HPC), gelatin, pregelatinized starch, polyvinylpyrrolidone, polyvinyl alcohol, pullulan, polyethylene glycol, natural gum, synthetic gum, copovidone, ethyl cellulose, methacrylate copolymer, and mixtures thereof, and may be hydroxypropylmethylcellulose, but is not limited thereto.

In the present invention, the wet granulation may be a high-speed shear granulation or a fluidized bed granulation, but is not limited thereto.

In the present invention, the pharmaceutically acceptable additives may be disintegrants, diluents, flavoring agents, sweetening agents, lubricants, or mixtures thereof, but are not limited thereto.

The disintegrants, diluents, flavoring agents, sweetening agents, lubricants, etc., are the same as those described in the foregoing item of the orally disintegrating tablet.

In the present invention, the sweetening agent may be used in the preparing wet granules, and may be further added as a pharmaceutically acceptable additive in the step of preparing a mixture.

The sweetening agent used in the preparing wet granules and the sweetening agent further added in the preparing a mixture may be of the same type or different types.

In the present invention, the sweetening agent used in the preparing wet granules may be referred to as a first sweetening agent, and the sweetening agent use in the preparing a mixture may be referred to as a second sweetening agent. In this case, “first,” “second,” etc. are only used to distinguish a plurality of components, and do not indicate priority thereof.

In the present invention, the compressing into tablets may be performed by using any tableting method commonly used in the art to which the present invention pertains.

The present invention may provide an orally disintegrating tablet prepared by the above preparation method.

Medicinal Use of Orally Disintegrating Tablet

The present invention may provide a method for preventing or treating diseases mediated by an acid pump antagonistic activity, which includes administering the orally disintegrating tablet.

The present invention may provide a use of the orally disintegrating tablet for preventing or treating diseases mediated by an acid pump antagonistic activity.

The present invention may provide a use of the orally disintegrating tablet in preparation of a drug for preventing or treating diseases mediated by an acid pump antagonistic activity.

The diseases mediated by an acid pump antagonistic activity may be at least one selected from the group consisting of gastrointestinal disease, gastroesophageal disease, gastroesophageal reflux disease (GERD), peptic ulcer, gastric ulcer, duodenal ulcer, NSAID-induced ulcer, gastritis, Helicobacter pylori infection, dyspepsia, functional dyspepsia, Zollinger-Ellison syndrome, nonerosive reflux disease (NERD), visceral referred pain, purosis, nausea, esophagitis, dysphagia, salivation, airway lesion and asthma, and specifically may be gastroesophageal reflux disease (GERD), but are not limited thereto.

In the medicinal use of the orally disintegrating tablet of the present invention, the contents described in the foregoing item of the orally disintegrating tablet may be equally applied as long as there is no contradiction. Thus, the wet granules, the components included therein, the content of respective components, the weight ratio of respective components, uses, effects, etc. of the foregoing orally disintegrating tablet may be directly applied to the medicinal use of the orally disintegrating tablet as long as there is no contradiction.

### Advantageous Effects of Invention

The orally disintegrating tablet of the present invention may disintegrate in the oral cavity within a short time and exhibit excellent sensory properties such as a masked bitter taste, etc., thereby greatly improving a patient's compliance with medication.

In addition, compared to the existing method for masking a bitter taste in which drug particles are coated with a polymeric substance or a lipid component or coated by using an enteric or gastric-soluble base, this preparation process is simple and economical, as well as exhibits a fast disintegration rate and an excellent release rate (speed) without any delay in drug disintegration and release, and produces a uniform tablet and small granular particles, thereby improving a patient's compliance with medication.

In addition, compared to the existing method for masking a bitter taste by simply adding various patterns of high sweetening agents and flavoring agents, this process show no change in appearance caused by moisture and heat (for example, occurrence of dark brown spots) on a surface of the tablet, thereby exhibiting excellent stability of appearance and produce a less total amount of related substances, thereby exhibiting excellent stability.

### BRIEF DESCRIPTION OF DRAWINGS

FIG. 1 is a view showing a drug dissolution rate at pH 1.2 with respect to the tablet prepared according to Example 1 and the K-CAP tablet.

FIG. 2 is a view showing a drug dissolution rate at pH 4.0 with respect to the tablet prepared according to Example 1 and the K-CAP tablet.

FIG. 3 is a view showing a change in properties of the tablets prepared according to Example 1 and Comparative Example 1 under stress and accelerated conditions.

### MODE FOR THE INVENTION

Hereinafter, the present invention will be described in more detail through exemplary embodiments. However, these exemplary embodiments are provided only for the purpose of illustrating the present invention, and thus the scope of the present invention is not limited thereto.

### Example 1: Preparation of Tegoprazan Orally Disintegrating Tablet 1

An orally disintegrating tablet was prepared according to the materials and contents described in Example 1 of table 1 below. Specifically, a binder solution was prepared by adding sucralose, a sweetening agent, to an aqueous solution of 58% (w/w) ethanol, followed by stirring for two hours or longer until completely dissolved. Tegoprazan, a main component, and mannitol 200SD, an excipient, were sieved through a 25-mesh sieve, mixed in a high-speed shear mixer, kneaded and granulated with the addition of the binder solution. The granulated product was dried in a fluidized bed dryer and subjected to a size regulation. Pearlitol flash, crospovidone CL-SF, maltitol, enzymatically modified stevia, peppermint flavor, and colloidal silicon dioxide were added to the size-regulated product as an excipient, disintegrant, sweetening agent, and flavoring agent, and then mixed and lubricated with the addition of magnesium stearate. After that, a tablet compression was performed by a conventional tableting method using a tableting machine. Each tablet was prepared to have a weight of 350 mg and hardness in the range of 5-8 kP.

### Example 2: Preparation of Tegoprazan Orally Disintegrating Tablet 2

An orally disintegrating tablet was prepared according to the materials and contents described in Example 2 of table 1 below. Specifically, a binder solution was prepared by adding hydroxypropyl methylcellulose (Pharmacoat 603), a binder, to an aqueous solution of 50% (w/w) ethanol to be completely dissolved, and then adding sucralose, a sweetening agent, followed by stirring for two hours or longer until completely dissolved. Tegoprazan, a main component, and mannitol 200SD, an excipient, were sieved through a 25-mesh sieve, added to a fluidized bed granulator (GPCG 1, manufactured by Glatt), prepared as granules by spraying the binder solution, and then subjected to size-regulation. Pearlitol flash, crospovidone CL-SF, maltitol, enzymatically modified stevia, peppermint flavor, and colloidal silicon dioxide were added to the size-regulated product as an excipient, disintegrant, sweetening agent, and flavoring agent, and then mixed and lubricated with the addition of magnesium stearate. After that, a tablet compression was performed by a conventional tableting method using a tableting machine. Each tablet was prepared to have a weight of 350.5 mg and hardness in the range of 5-8 kP.

### Example 3: Preparation of Tegoprazan Orally Disintegrating Tablet 3

An orally disintegrating tablet was prepared according to the materials and contents described in Example 3 of table 1 below. Specifically, a binder solution was prepared by adding maltitol, a sweetening agent, to an aqueous solution of 50% (w/w) ethanol, followed by stirring for two hours or longer until completely dissolved. Tegoprazan, a main component, and mannitol 2005D, an excipient, were sieved through a 25-mesh sieve, mixed in a high-speed shear mixer, kneaded and granulated with the addition of the binder solution. The granulated product was dried in a fluidized bed dryer and subjected to a size regulation. Pearlitol flash, crospovidone CL-SF, maltitol, enzymatically modified stevia, peppermint flavor, and colloidal silicon dioxide were added to the size-regulated product as an excipient, disintegrant, sweetening agent, and flavoring agent, and then mixed and lubricated with the addition of magnesium stearate. After that, a tablet compression was performed by a conventional tableting method using a tableting machine. Each tablet was prepared to have a weight of 350 mg and hardness in the range of 5-8 kP.

### Example 4: Preparation of Tegoprazan Orally Disintegrating Tablet 4

An orally disintegrating tablet was prepared according to the materials and contents described in Example 4 of table 1 below. Specifically, a binder solution was prepared by adding aspartame, a sweetening agent, to an aqueous solution of 50% (w/w) ethanol, followed by stirring for two hours or longer until completely dissolved. Tegoprazan, a main component, and mannitol 2005D, an excipient, were sieved through a 25-mesh sieve, mixed in a high-speed shear mixer, kneaded and granulated with the addition of the binder solution. The granulated product was dried in a fluidized bed dryer and subjected to a size regulation. Pearlitol flash, crospovidone CL-SF, maltitol, enzymatically modified stevia, peppermint flavor, and colloidal silicon dioxide were added to the size-regulated product as an excipient, disintegrant, sweetening agent, and flavoring agent, and then mixed and lubricated with the addition of magnesium stearate. After that, a tablet compression was performed by a conventional tableting method using a tableting machine. Each tablet was prepared to have a weight of 350 mg and hardness in the range of 5-8 kP.

### Example 5: Preparation of Tegoprazan Orally Disintegrating Tablet 5

An orally disintegrating tablet was prepared according to the materials and contents described in Example 5 of table 1 below. Specifically, a binder solution was prepared by adding sucralose, a sweetening agent, to a 50% (w/w) aqueous solution of ethanol, followed by stirring for two hours or longer until completely dissolved. Tegoprazan, a main component, and xylitol, an excipient, were sieved through a 25-mesh sieve, mixed in a high-speed shear mixer, kneaded and granulated with the addition of the binder solution. The granulated product was dried in a fluidized bed dryer and subjected to a size regulation. Pearlitol flash, crospovidone CL-SF, maltitol, enzymatically modified stevia, peppermint flavor, and colloidal silicon dioxide were added to the size-regulated product as an excipient, disintegrant, sweetening agent, and flavoring agent, and then mixed and lubricated with the addition of magnesium stearate. After that, a tablet compression was performed by a conventional tableting method using a tableting machine. Each tablet was prepared to have a weight of 350 mg and hardness in the range of 5-8 kP.

### Comparative Example 1. Preparation of Tegoprazan Orally Disintegrating Tablet 6

An orally disintegrating tablet was prepared according to the materials and contents described in Comparative Example 1 of table 1 below. Specifically, all the materials described in Comparative Example 1 of table 1 were sieved through a 25-mesh sieve, mixed and subjected to a tablet compression by a conventional tableting method using a tableting machine. Each tablet was prepared to have a weight of 350 mg and hardness in the range of 5-8 kP.

TABLE 1

| | | Example 1 | | Example 3 | Example 4 | Example 5 | Comparative |
|---|---|---|---|---|---|---|---|
| | | High- | Example 2 | High- | High- | High- | Example 1 |
| | | speed | Fluidized | speed | speed | speed | Direct |
| | | shear | bed | shear | shear | shear | tableting |
| Classification | | granulation | granulation | granulation | granulation | granulation | method |
| Composition | | Usage | Usage | Usage | Usage | Usage | Usage |
| | | (mg/T) | (mg/T) | (mg/T) | (mg/T) | (mg/T) | (mg/T) |
| Pre-mix | Tegoprazan | 50.0 | 50.0 | 50.0 | 50.0 | 50.0 | 50.0 |
| portion | Mannitol | 50.0 | 50.0 | 50.0 | 50.0 | — | 50.0 |
| | (200SD) | | | | | | |
| | Xylitol | — | — | — | — | 50.0 | — |
| Binder | Sucralose | 3.5 | 3.5 | — | — | 3.5 | — |
| solution | Maltitol | — | — | 3.5 | — | — | — |
| | Aspartame | — | — | — | 3.5 | — | — |
| | HPMC | — | 0.5 | — | — | — | — |
| | Purified water | 3.6 | 75.0 | 7.5 | 7.5 | 7.5 | — |
| | Ethanol | 5.0 | 75.0 | 7.5 | 7.5 | 7.5 | — |
| Post-mix | Pearlitol flash | 192.3 | 195.0 | 195.0 | 195.0 | 195.0 | 195.0 |
| portion | Crospovidone | 17.5 | 17.0 | 17.0 | 17.0 | 17.0 | 17.0 |
| | Maltitol | 17.5 | 17.0 | 17.0 | 17.0 | 17.0 | 17.0 |
| | Sucralose | — | — | — | — | — | 3.5 |
| | Enzymatically | 1.8 | 1.5 | 1.5 | 1.5 | 1.5 | 1.5 |
| | modified | | | | | | |
| | stevia | | | | | | |
| | Peppermint | 3.5 | 3.0 | 3.0 | 3.0 | 3.0 | 3.0 |
| | flavor | | | | | | |
| | Colloidal | 3.5 | 3.0 | 3.0 | 3.0 | 3.0 | 3.0 |
| | silicon dioxide | | | | | | |
| | Magnesium | 10.5 | 10.0 | 10.0 | 10.0 | 10.0 | 10.0 |
| | stearate | | | | | | |
| | Total weight | 350.0 | 350.5 | 350.0 | 350.0 | 350.0 | 350.0 |

### Experimental Example 1: Evaluation of Disintegration Time

An orally disintegrating tablet needs to be administered while disintegrating in the oral cavity, and thus rapid disintegration is required. Thus, a disintegration time of the tablets prepared according to Examples 1 to 5 and Comparative Example 1 was measured.

For an in vitro disintegration experiment, a filter paper having a diameter of 90 mm

(see WH1442090) was placed on a petri dish (100×10 mm), and then 10 mL of an aqueous solution of 10% (w/w) cobalt(II) chloride hexahydrate was completely soaked into the filter paper of the dish, after which a tablet was placed thereon so as to measure a time taken until water reached to an end surface of the tablet due to a capillary action by visually checking a change in colors with naked eyes (measured three times).

For an in vivo disintegration experiment, ten subjects were asked to take the tablet without water, so as to measure a time taken until the tablet is completely disintegrated by saliva in the oral cavity.

For a disintegration experiment in a disintegration tester, a measurement was made according to a disintegration test method of the Korean Pharmacopoeia general test method (n=6).

An average value of each test is shown in table 2 below.

TABLE 2

| | Hardness | | | Disintegration |
|---|---|---|---|---|
| No. | (kP) | in vitro | in vivo | tester |
| Example 1 | 6 | 17 sec | 22 sec | 19 sec |
| Example 2 | 6 | 16 sec | 22 sec | 23 sec |
| Example 3 | 6 | 17 sec | 21 sec | 22 sec |
| Example 4 | 6 | 18 sec | 22 sec | 20 sec |
| Example 5 | 6 | 19 sec | 22 sec | 22 sec |
| Comparative | 6 | 18 sec | 20 sec | 23 sec |
| Example 1 | | | | |

As a result, it was confirmed that the tablets prepared according to Examples 1 to 5 and Comparative Example 1 disintegrate rapidly within 30 seconds in the in vitro disintegration experiment, the in vivo disintegration experiment, and the disintegration experiment in the disintegration tester all.

### Experimental Example 2: Sensory Evaluation

Ten subjects were asked to take the tablets prepared according to above Examples 1 to 5 and Comparative Example 1 without water, after which a sensory evaluation (for a feeling of irritation and a bitter taste) was performed. Each subject recorded a score (0-5 points) for the feeling of irritation and the bitter taste, and the resulting scores were averaged and shown in table 3 below.

TABLE 3

| | Feeling of | | |
|---|---|---|---|
| No. | irritation¹⁾ | Bitter taste²⁾ | Feeling in the mouth |
| Example 1 | 1.1 | 1.0 | Gently disintegrates |
| Example 2 | 1.3 | 1.3 | Gently disintegrates |
| Example 3 | 1.8 | 1.8 | Gently disintegrates |
| Example 4 | 1.7 | 1.9 | Gently disintegrates |
| Example 5 | 1.8 | 1.5 | Gently disintegrates |
| Comparative | 1.3 | 4.6 | Unpleasant feeling |
| Example 1 | | | in the mouth and a |
| | | | strong bitter taste |

¹⁾Feeling of irritation: Very much-5 points; slightly much-4 points; Average-3 points; slightly less-2 points; very little-1 point; None-0 points
²⁾Bitter taste: Very much-5 points; slightly much-4 points; Average-3 points; slightly less-2 points; very little-1 point; None-0 points

As a result, it was confirmed that Comparative Example 1 prepared with the direct

tableting method of adding the sweetening agent in a powder form has a very strong bitter taste and an unpleasant feeling in the mouth, while Examples 1 and 3 to 5 prepared with the high-speed shear granulation and Example 2 prepared with the fluidized bed granulation have a very little bitter taste and a feeling of smooth disintegration in the oral cavity as the binder solution including the sweetening agent attaches and coats the particles of tegoprazan and excipients, thereby showing an effect on alleviating the bitter taste as well as excellent sensory properties.

### Experimental Example 3: Dissolution Rate Evaluation

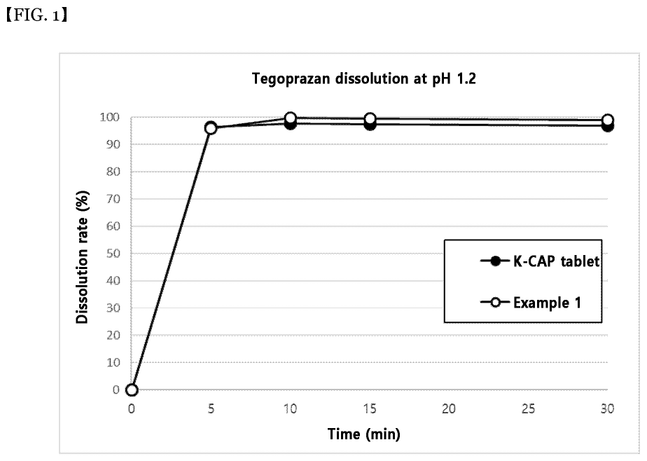

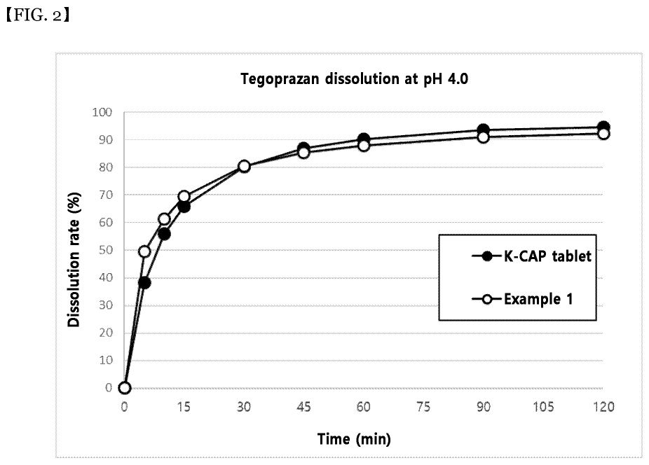

A dissolution rate of the drug was compared between the tablet prepared according to above Example 1 and the 50 mg K-CAP tablet of HK Innoen Co., Ltd., on the basis of Chapter 3 Comparative Dissolution Test of Pharmaceutical Equivalence Test Standards. Upon starting the test, samples of the dissolution test solution at pH 1.2 were collected at 0, 5, 10, 15 and 30 minutes and those at pH 4.0 were collected at 0, 5, 10, 15, 30, 45, 60, 90 and 120 minutes and subjected to a liquid chromatography under the following conditions, after which the dissolution rates of tegoprazan were calculated and shown in Table 4 and FIGS. 1 and 2 below.

<Dissolution Conditions>
Number of rotations: 50 rotations/min
Amount of test solution: 900 mL
Temperature of test solution: 37±0.5° C.
Test solution: Solutions at pH 1.2 and pH 4.0 of Korean Pharmacopoeia

<Liquid Chromatography Conditions>
Column: A column filled with octadecylsilylated silica gel for liquid chromatography with a particle diameter of 5 μm in a stainless tube with an inner diameter of about 4.6 mm and a length of 15 cm
Column temperature: Constant temperature around 30° C.
Amount of sample injection: 10 μL
Mobile phase: 0.01 mol/L ammonium acetate buffer: ACN=11:9
Flow rate: 1.0 mL/min
Detector: Ultraviolet absorbance photometer (measurement wavelength of 262 nm)

TABLE 4

| | pH 1.2 dissolution rate (%) | | pH 4.0 dissolution rate (%) | |
|---|---|---|---|---|
| Time | | K-CAP tablet | | K-CAP tablet |
| (minute) | Example 1 | 50 mg | Example 1 | 50 mg |
| 0 | 0 | 0 | 0 | 0 |
| 5 | 95.7 | 96.2 | 49.5 | 38.2 |
| 10 | 99.8 | 97.6 | 61.3 | 56.0 |
| 15 | 99.5 | 97-3 | 69.5 | 65.9 |
| 30 | 98.8 | 96.8 | 80.5 | 80.3 |
| 45 | | | 85.3 | 86.8 |
| 60 | | | 87.9 | 90.3 |
| 90 | | | 90.9 | 93-5 |
| 120 | | | 92.3 | 94.6 |

As a result, it was confirmed that the tablet prepared according to Example 1 exhibits an excellent dissolution rate of tegoprazan at a level similar to that of the control drug K-CAP tablet without any delay in release.

### Experimental Example 4: Stability Evaluation

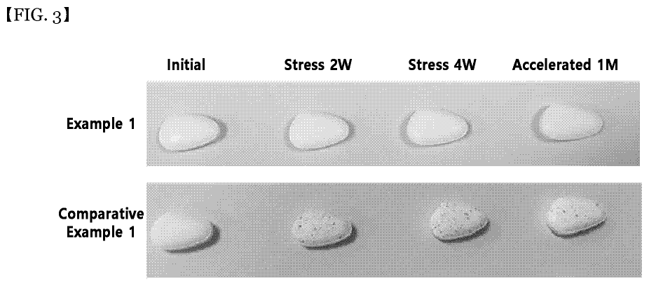

In order to evaluate the stability under stress and accelerated conditions of Example 1 and Comparative Example 1, a stability test was performed in the HDPE bottles and PTP (aluminum) packaging materials under stress conditions (60° C., 80% RH) and accelerated conditions (40° C., 75% RH), and the total amount of related substances and the change in appearance thereof are shown in Table 6 and FIG. 3 below, respectively.

<Liquid Chromatography Conditions>
Column: A column filled with octadecylsilylated silica gel for liquid chromatography with a particle diameter of 2.7 μcm in a stainless tube with an inner diameter of about 4.6 mm and a length of 15 cm
Column temperature: Constant temperature around 30° C.
Amount of sample injection: 10 μL
Mobile phase A: 0.01 mol/L ammonium acetate buffer: ACN=19:1
Mobile phase B: ACN
Flow rate: 0.8 mL/min
Detector: Ultraviolet absorbance photometer (measurement wavelength of 220 nm)

TABLE 5
Mobile phase gradient conditions

| | Mobile phase | |
|---|---|---|
| Time (minute) | A (%) | B (%) |
| 0~2 | 88 | 12 |
| 2~3 | 88 → 70 | 12 → 30 |
| 3~15 | 70 | 30 |
| 15~16 | 70 → 20 | 30 → 80 |
| 16~17 | 20 → 10 | 80 → 90 |
| 17~20 | 10 | 90 |
| 20~20.01 | 10 → 88 | 90 → 12 |
| 20.01~25 | 88 | 12 |

TABLE 6

| No. | | | | |
|---|---|---|---|---|
| Total related | Example 1 | | Comparative Example 1 | |
| substance | HDPE Bottle | Al/Al PTP | HDPE Bottle | Al/Al PTP |
| Initial | 0.12 | 0.12 | 0.15 | 0.15 |
| Stress week 2 | 0.12 | 0.12 | 0.27 | 0.18 |
| Stress week 4 | 0.13 | 0.13 | 0.39 | 0.22 |
| Accelerated 1 | 0.12 | 0.12 | 0.15 | 0.15 |
| month | | | | |

As a result, it was confirmed that the tablet prepared according to Example 1 produces a less total amount of related substances under stress and accelerated conditions compared to the tablet prepared according to Comparative Example 1 (Table 6), and the tablet of Comparative Example 1 produces a black-brown spot on a tablet surface under stress and accelerated conditions, while the tablet prepared according to Example 1 shows no change in appearance under stress and accelerated conditions (FIG. 3), thereby exhibiting excellent stability.

### Experimental Example 5: Stability Evaluation According to Sweetening Agent Type and Weight Ratio

In order to evaluate stability under stress conditions according to the type of sweetening agent included in the granules and the weight ratio of tegoprazan and sweetening agent in the granules, an orally disintegrating tablet was prepared according to the materials and contents described in table 7 below (Examples 6 to 10 and Comparative Example 2 were prepared by the same method as in Example 1), and thus a stability test was performed under stress conditions (60° C., 80% RH).

The total amount of related substances is shown in table 8 below.

TABLE 7

| | | | | | Comparative | | |
|---|---|---|---|---|---|---|---|
| Classification | | Example 6 | Example 7 | Example 8 | Example 2 | Example 9 | Example 10 |
| Tegoprazan:sweetening | | 1:0.05 | 1:0.1 | 1:0.2 | 1:0.5 | 1:0.2 | 1:0.2 |
| agent | | | | | | | |
| Composition | | Usage | Usage | Usage | Usage | Usage | Usage |
| | | (mg/T) | (mg/T) | (mg/T) | (mg/T) | (mg/T) | (mg/T) |
| Pre-mix | Tegoprazan | 50.0 | 50.0 | 50.0 | 50.0 | 50.0 | 50.0 |
| portion | Mannitol | 50.0 | 50.0 | 50.0 | 50.0 | 50.0 | 50.0 |
| | (200SD) | | | | | | |
| Binder | Sucralose | 2.5 | 5.0 | 10.0 | 25.0 | — | — |
| solution | Aspartame | 1 | 1 | — | — | 10.0 | — |
| | Maltitol | — | — | — | — | — | 10.0 |
| | Purified water | 7.5 | 7.5 | 7.5 | 7.5 | 7.5 | 7.5 |
| | Ethanol | 7.5 | 7.5 | 7.5 | 7.5 | 7.5 | 7.5 |
| Post-mix | Pearlitol flash | 192.3 | 192.3 | 192,3 | 192.3 | 192.3 | 192.3 |
| portion | Crospovidone | 17.5 | 17.5 | 17.5 | 17.5 | 17.5 | 17.5 |
| | Maltitol | 17.5 | 17.5 | 17.5 | 17.5 | 17.5 | 17.5 |
| | Enzymatically | 1.8 | 1.8 | 1.8 | 1.8 | 1.8 | 1.8 |
| | modified stevia | | | | | | |
| | Peppermint | 3.5 | 3.5 | 3.5 | 3.5 | 3.5 | 3.5 |
| | flavor | | | | | | |
| | Colloidal silicon | 3.5 | 3.5 | 3.5 | 3.5 | 3.5 | 3.5 |
| | dioxide | | | | | | |
| | Magnesium | 10.5 | 10.5 | 10.5 | 10.5 | 10.5 | 10.5 |
| | stearate | | | | | | |
| | Total weight | 349.0 | 351.5 | 356.5 | 371.5 | 356.5 | 356.5 |

TABLE 8

| | | | | Comparative | | |
|---|---|---|---|---|---|---|
| Classification | Example 6 | Example 7 | Example 8 | Example 2 | Example 9 | Example 10 |
| Tegoprazan:sucralos | 1:0.05 | 1:0.1 | 1:0.2 | 1:0.5 | — | — |
| Tegoprazan:aspartame | — | — | — | — | 1:0.2 | — |
| Tegoprazan:maltitol | — | — | — | — | — | 1:0.2 |
| Initial | 0.08 | 0.08 | 0.08 | 0.08 | 0.10 | 0.10 |
| Stress week 2 | 0.08 | 0.08 | 0.08 | 0.99 | 0.11 | 0.10 |
| Stress week 4 | 0.08 | 0.08 | 0.08 | 1.91 | 0.11 | 0.10 |

As a result, it was confirmed that a less total amount of related substances are

produced from the tablets of Examples 6 to 10 in which the weight ratio of tegoprazan and the sweetening agent is 1:0.05 to 1:0.2 compared to the tablet of Comparative Example 2 in which the weight ratio of tegoprazan and the sweetening agent is 1:0.5, thereby exhibiting excellent stability.

Meanwhile, according to the type of the sweetening agent, it was confirmed that the tablet of Example 8 having sucralose, the tablet of Example 9 having aspartame, and the tablet of Example 10 having maltitol all produce a less total amount of related substances, thereby exhibiting excellent stability regardless of the type of sweetening agent.

While specific portions of the present invention have been described in detail above,

it is apparent to those skilled in the art that such detailed descriptions are set forth to illustrate exemplary embodiments only, but are not construed to limit the scope of the present invention. Thus, it should be understood that the substantial scope of the present invention is defined by the accompanying claims and equivalents thereto.

1. An orally disintegrating tablet comprising wet granules including a compound represented by formula 1 below, a pharmaceutically acceptable salt thereof, a hydrate or solvate thereof, or a mixture thereof;
and a sweetening agent:

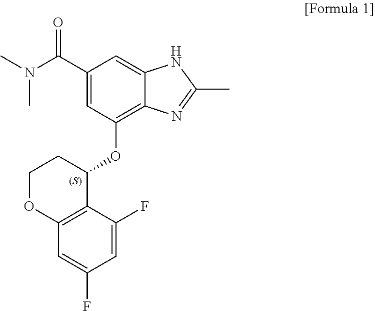

2. The orally disintegrating tablet of claim 1, wherein the wet granules comprise the compound represented by formula 1, a pharmaceutically acceptable salt thereof, a hydrate or solvate thereof, or a mixture thereof, and the sweetening agent at a weight ratio of 1:0.001 to 1:0.4.

3. The orally disintegrating tablet of claim 2, wherein the wet granules comprise the compound represented by formula 1, a pharmaceutically acceptable salt thereof, a hydrate or solvate thereof, or a mixture thereof, and the sweetening agent at a weight ratio of 1:0.05 to 1:0.2.

4. The orally disintegrating tablet of claim 1, wherein the sweetening agent is one selected from the group consisting of sucralose, aspartame, saccharin, acesulfame potassium, stevioside, enzymatically modified stevia, sucrose, isomalt, maltitol, mannitol, sorbitol, steviol glycoside, erythritol, xylose, xylitol, lactitol, neotame, ribose, tomatine, polyglycitol, advantam, tagatose, trehalose, glucose, maltose, dextrose, white sugar, fructose, honey, glycyrrhizin, monellin, rubusoside, curculin, corn syrup, lactose, oligosaccharides, and mixtures thereof.

5. The orally disintegrating tablet of claim 1, wherein the sweetening agent is comprised in an amount of 0.01 to 10 wt % based on the total weight of the tablet.

6. The orally disintegrating tablet of claim 1, wherein the orally disintegrating tablet further comprises a disintegrant.

7. The orally disintegrating tablet of claim 6, wherein the disintegrant is one selected from the group consisting of crospovidone, croscarmellose sodium, sodium starch glycolate, alginic acid, sodium alginate, low-substituted hydroxypropyl cellulose, carboxymethyl cellulose, corn starch, pregelatinized starch, microcrystalline cellulose, hydroxypropyl cellulose, sodium bicarbonate, and mixtures thereof.

8. The orally disintegrating tablet of claim 6, wherein the disintegrant is comprised in an amount of 1 to 50 wt % based on the total weight of the tablet.

9. The orally disintegrating tablet of claim 1, wherein the orally disintegrating tablet further comprises diluents, flavoring agents, sweetening agents, lubricants, or mixtures thereof.

10. The orally disintegrating tablet of claim 9, wherein the diluent is one selected from the group consisting of mannitol, lactose, starch, micro-crystalline cellulose, ludipress, pearlitol flash, calcium dihydrogen phosphate, dextrose, maltose, erythritol, sucrose, maltitol, trihalose, glucose, xylitol, F-melt, sorbitol, pregelatinized starch, anhydrous calcium hydrogen phosphate, dicalcium phosphate, and mixtures thereof.

11. The orally disintegrating tablet of claim 9, wherein the diluent is comprised in an amount of 1 to 99 wt % based on the total weight of the tablet.

12. The orally disintegrating tablet of claim 9, wherein the flavoring agent is one selected from the group consisting of peppermint flavor, yogurt flavor, fruit flavor, and mixtures thereof.

13. The orally disintegrating tablet of claim 9, wherein the flavoring agent is comprised in an amount of 0.01 to 10 wt % based on the total weight of the tablet.

14. The orally disintegrating tablet of claim 9, wherein the lubricant is one selected from the group consisting of stearic acid, stearic acid metal salts, talc, colloidal silica, sucrose fatty acid ester, hydrogenated vegetable oil, wax, glycerin fatty acid ester, glycerol dibehenate, and mixtures thereof.

15. The orally disintegrating tablet of claim 9, wherein the lubricant is comprised in an amount of 0.1 to 10 wt % based on the total weight of the tablet.

16. The orally disintegrating tablet of claim 1, wherein the wet granules are prepared with a binder solution including one selected from the group consisting of alcohol, water, and a mixture thereof and a sweetening agent.

17. The orally disintegrating tablet of claim 16, wherein the binder solution further comprises a binder or an additive capable of giving binding force.

18. The orally disintegrating tablet of claim 17, wherein the binder or the additive capable of giving binding force is one selected from the group consisting of hydroxypropyl methylcellulose (HPMC), hydroxypropyl cellulose (HPC), gelatin, pregelatinized starch, polyvinylpyrrolidone, polyvinyl alcohol, pullulan, polyethylene glycol, natural gum, synthetic gum, copovidone, ethyl cellulose, methacrylate copolymer, and mixtures thereof.

19. The orally disintegrating tablet of claim 1, wherein the wet granules are prepared by high-speed shear granulation or fluidized bed granulation.

20. The orally disintegrating tablet of claim 1, wherein the orally disintegrating tablet disintegrates within 30 seconds.

21. The orally disintegrating tablet of claim 1, wherein the orally disintegrating tablet has a masked bitter taste.

22. The orally disintegrating tablet of claim 1, wherein the orally disintegrating tablet is used for preventing or treating diseases mediated by an acid pump antagonistic activity.

23. The orally disintegrating tablet according to claim 22, wherein the diseases mediated by an acid pump antagonistic activity are at least one selected from the group consisting of gastrointestinal disease, gastroesophageal disease, gastroesophageal reflux disease (GERD), peptic ulcer, gastric ulcer, duodenal ulcer, NSAID-induced ulcer, gastritis, Helicobacter pylori infection, dyspepsia, functional dyspepsia, Zollinger-Ellison syndrome, nonerosive reflux disease (NERD), visceral referred pain, purosis, nausea, esophagitis, dysphagia, salivation, airway lesion and asthma.

24. A method for preparing an orally disintegrating tablet, comprising:
(1) preparing wet granules including a compound represented by formula 1 below, a pharmaceutically acceptable salt thereof, a hydrate or solvate thereof, or a mixture thereof, and a sweetening agent;
(2) preparing a mixture by adding one or more pharmaceutically acceptable additives to the wet granules; and
(3) compressing the mixture into tablets:

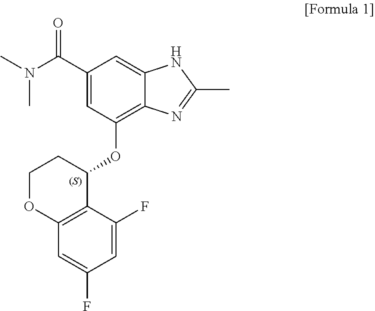

25. The method of claim 24, wherein the sweetening agent is one selected from the group consisting of sucralose, aspartame, saccharin, acesulfame potassium, stevioside, enzymatically modified stevia, sucrose, isomalt, maltitol, mannitol, sorbitol, steviol glycoside, erythritol, xylose, xylitol, lactitol, neotame, ribose, tomatine, polyglycitol, advantam, tagatose, trehalose, glucose, maltose, dextrose, white sugar, fructose, honey, glycyrrhizin, monellin, rubusoside, curculin, corn syrup, lactose, oligosaccharides, and mixtures thereof.

26. The method of claim 24, wherein the preparing of wet granules is performed by a wet granulation with a binder solution including one selected from the group consisting of alcohol, water, and a mixture thereof and a sweetening agent.

27. The method of claim 26, wherein the binder solution further comprises a binder or an additive capable of giving binding force.

28. The method of claim 27, wherein the binder or the additive capable of giving binding force is one selected from the group consisting of hydroxypropyl methylcellulose (HPMC), hydroxypropyl cellulose (HPC), gelatin, pregelatinized starch, polyvinylpyrrolidone, polyvinyl alcohol, pullulan, polyethylene glycol, natural gum, synthetic gum, copovidone, ethyl cellulose, methacrylate copolymer, and mixtures thereof.

29. The method of claim 26, wherein the wet granulation is a high-speed shear granulation or a fluidized bed granulation.

30. The method of claim 24, wherein the pharmaceutically acceptable additives are disintegrants, diluents, flavoring agents, sweetening agents, lubricants, or mixtures thereof.

31. An orally disintegrating tablet prepared by the preparation method according to any one of claims 24 to 30.

32. A method for preventing or treating diseases mediated by an acid pump antagonistic activity, which comprises administering the orally disintegrating tablet according to any one of claims 1 to 23.

33. A use of the orally disintegrating tablet according to any one of claims 1 to 23 for preventing or treating diseases mediated by an acid pump antagonistic activity.

34. A use of the orally disintegrating tablet according to any one of claims 1 to 23 in preparation of a drug for preventing or treating diseases mediated by an acid pump antagonistic activity.
